(19) United States
(12) Patent Application Publication
(10) Pub. No.: US 2025/0154358 A1
(43) Pub. Date: May 15, 2025

(54) ORGANIC DYE COMPOSITION, AND SPRAY AGENT COMPRISING SAME

(71) Applicant: PANASONIC INTELLECTUAL PROPERTY MANAGEMENT CO., LTD., Osaka (JP)

(72) Inventor: Mari UEDA, Osaka (JP)

(73) Assignee: PANASONIC INTELLECTUAL PROPERTY MANAGEMENT CO., LTD., Osaka (JP)

(21) Appl. No.: 19/021,611

(22) Filed: Jan. 15, 2025

Related U.S. Application Data
(63) Continuation of application No. PCT/JP2023/026696, filed on Jul. 20, 2023.

(30) Foreign Application Priority Data
Jul. 29, 2022 (JP) 2022-121783

(51) Int. Cl. C09B 11/08; C09B 19/00; G01N 21/64

(52) U.S. Cl. CPC C09B 11/08; C09B 19/00; G01N 21/6428; G01N 2021/6439

(57) ABSTRACT

One aspect of the present invention relates to an organic dye composition containing an organic dye (A) that is discolored by hydrolysis and emits fluorescence by excitation light having a wavelength of 400 nm or more and 550 nm or less, an alcohol (B), and water (C), in which a content rate of the organic dye (A) is 1 μmol/L or more, and a viscosity is 5 mPa·s or more and 45 mPa·s or less.

### FILED OF INVENTION

The present invention relates to an organic dye composition and a spray agent containing the same.

### BACKGROUND ART

With public health assumptions shifting from “safe” to “dangerous”, the threat of invisible microorganisms has never been greater. Disinfection is performed at a human hand and every place where a human hand touches, but since a measure of the disinfection effect is invisible, there is a problem that it is difficult to understand an appropriate level of disinfection measures. There are individual differences in the risk of invisible microorganisms, and disinfection measures may be insufficient depending on a person, or excessive measures may be taken more than necessary for a person with great anxiety. Therefore, there is a demand for a practical tool that simply shows the effect of measures such as disinfection.

As a method for measuring cleanliness and detecting microorganisms, a method using a luminescent reagent, a fluorescent indicator, or the like has been known. For example, Patent Literature 1 discloses that adenosine phosphate esters in a sample obtained by wiping the sample from a measurement target are caused to emit light by an action of the sample and a luminescent reagent, and the amount of emitted light is measured to measure the degree of contamination and the degree of cleanliness of the measurement target, and the degree of cleanliness is evaluated. In addition, Patent Literature 2 describes a method for detecting the presence of bacterial spores of microorganisms by culturing a sample and measuring the microbial metabolic activity in the sample using a metabolic dye or the like. Furthermore, Patent Literature 3 reports a method for detecting viable bacteria, including detecting a light spot generated by bringing a microbial cell sample into contact with a fluorescent indicator of an intracellular esterase activity index or the like and irradiating excitation light.

However, in the technique described in Patent Literature 1, since a sample is obtained by wiping off the measurement target, the microorganism concentration can be grasped only in the wiped region. Therefore, it is not possible to see the appropriateness over a wide range of disinfection effects.

In addition, in the technique described in Patent Literature 2, in addition to the problem of locality that the microorganism concentration can be grasped only in the sampled region, a step of culturing microorganisms contained in a specimen is required, and it takes time until the result is obtained, which is not practical.

Moreover, in the technique described in Patent Literature 3, a step of collecting microbial cells using a membrane filter is required, and it takes time to prepare a specimen.

The present invention is made in view of such circumstances, and it is a main object of the present invention to provide a practical tool capable of easily checking the disinfection effect and the presence or absence of microorganisms in a wide range in a short time.

Patent Literature 1: JP 2003-35673 A

Patent Literature 2: JP 2020-530286 A

Patent Literature 3: JP 2006-238779 A

### SUMMARY OF THE INVENTION

As a result of intensive studies, the present inventor found out that the problems can be solved by an organic dye composition having the following configuration, and completed the present invention by conducting further studies based on this finding.

That is, an organic dye composition related to one aspect of the present invention contains an organic dye (A) that is discolored by hydrolysis and emits fluorescence by excitation light having a wavelength of 400 nm or more and 550 nm or less, an alcohol (B), and water (C), in which a content rate of the organic dye (A) is 1 μmol/L or more, and a viscosity is 5 mPa·s or more and 45 mPa·s or less.

In addition, a spray agent according to another aspect of the present invention contains the organic dye composition.

### BRIEF DESCRIPTION OF THE DRAWINGS

FIG. 1 is a block diagram showing an example of a microorganism visualization system using an organic dye composition of the present embodiment.

FIGS. 2A and 2B are block diagrams showing another example of the microorganism visualization system using the organic dye composition of the present embodiment.

FIG. 3 is a block diagram showing still another example of the microorganism visualization system using the organic dye composition of the present embodiment.

### DETAILED DESCRIPTION

Hereinafter, embodiments according to the present invention will be specifically described, but the present invention is not limited thereto.

### Organic Dye Composition

The organic dye composition of the present embodiment contains an organic dye (A), an alcohol (B), and water (C). In the organic dye composition, the content rate of the organic dye (A) is 1 μmol/L or more, and the viscosity is 5 mPa·s or more and 45 mPa·s or less. The organic dye (A) is discolored by hydrolysis, and emits fluorescence by excitation light having a wavelength of 400 nm or more and 550 nm or less.

Such an organic dye composition is useful as a spray agent by having a viscosity in the above range, and can be sprayed in a wide range. In addition, the organic dye composition of the present embodiment contains the organic dye (A) in an amount of 1 μmol/L or more, thus when the organic dye composition comes into contact with microorganisms, the organic dye composition causes hydrolysis and is discolored, and emits fluorescence with excitation light having a wavelength of 400 nm or more and 550 nm or less. First, the presence or absence of microorganisms can also be confirmed by visually recognizing discoloration due to the hydrolysis. Furthermore, although depending on the concentration of microorganisms, the organic dye (A) usually emits light about 1 to 15 minutes after spraying. Therefore, for example, by applying a green or red LED light or the like to the sprayed location, the presence or absence of microorganisms can be quickly and easily visually recognized in a wide range. Therefore, with the organic dye composition of the present embodiment, the disinfection effect and the presence or absence of microorganisms in a wide range can be easily checked in a short time. In addition, since detection by light irradiation is very sensitive, microorganisms can be detected even in a microorganism low-concentration region where the discoloration cannot be visually confirmed.

Therefore, according to the present embodiment, it is possible to provide an organic dye composition that can be used as a practical tool capable of easily checking the disinfection effect and the presence or absence of microorganisms in a wide range in a short time, and a spray agent using the same.

When the organic dye composition is sprayed, the viscosity of the organic dye composition is preferably further 8 mPa·s or more and 45 mPa·s or less from the viewpoint that splashing and rolling of droplets from an object to be sprayed can be reduced and fixability can be improved.

Hereinafter, each component contained in the organic dye composition will be described.

### Organic Dye (A)

As described above, the organic dye (A) of the present embodiment is an organic dye that is discolored by hydrolysis and emits fluorescence by excitation light having a wavelength of 400 nm or more and 550 nm or less. The phrase “discolored by hydrolysis” means that when the organic dye (A) comes into contact with microorganisms, the organic dye (A) is hydrolyzed by an enzymatic reaction in the microbial cell, resulting in a molecular structure exhibiting a color different from that of the dye molecule before being hydrolyzed. That is, the organic dye (A) is a dye whose molecular structure changes and whose absorption spectrum in the visible light region changes when the organic dye (A) comes into contact with microorganisms.

In the present specification, the phrase “emits fluorescence by excitation light having a wavelength of 400 nm or more and 550 nm or less” means that red fluorescence and red are exhibited at an excitation wavelength of about 400 to 500 nm, or green fluorescence and green are exhibited at an excitation wavelength of about 450 to 550 nm.

As the organic dye (A), any organic dye may be used as long as it has the above-described characteristics, and for example, an organic dye for staining living cells or the like can be used. More specifically, for example, it is preferably at least one selected from compounds represented by the following Formulas (1) to (5).

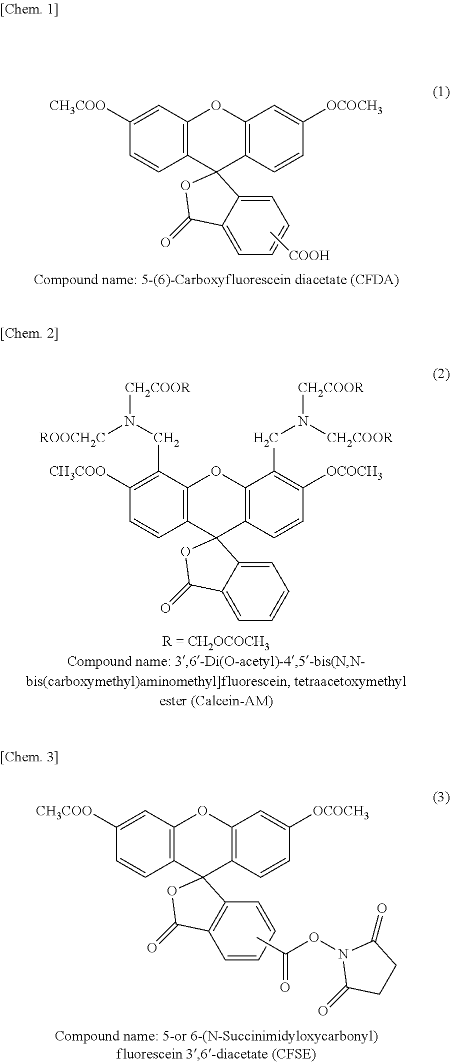

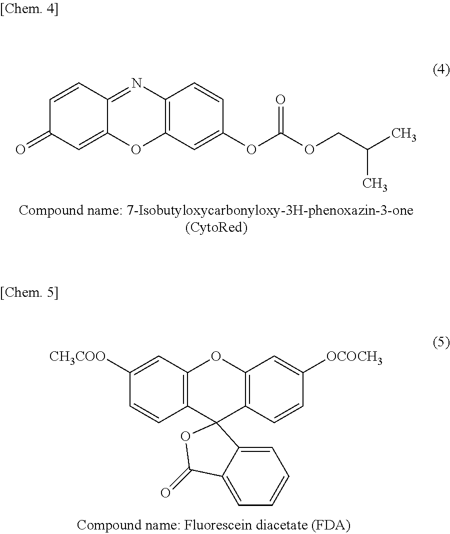

Organic dyes as described above can be used safely because they exhibit esterase activity and are not toxic.

The organic dyes (A) can be used singly or in combination of two or more kinds thereof.

In the organic dye composition of the present embodiment, the content rate of the organic dye (A) is 1 μmol/L or more. When the content rate of the organic dye (A) is in the above range, detection of microorganisms and the like can be more reliably performed. More preferably, the content rate of the organic dye (A) is 10 μmol/L or more. In addition, it is not necessary to particularly set an upper limit on the content rate, but the content rate is preferably 100 μmol/L or less from the viewpoint of further suppressing cytotoxicity.

### Alcohol (B)

The organic dye composition of the present embodiment contains an alcohol (B) in order to impart moisture-retaining properties and thickening properties to the composition. By containing the alcohol (B), the viscosity in the organic dye composition the organic dye composition of the present embodiment can be 5 mPa·s or more and 45 mPa·s or less. The alcohol (B) also plays a role of fixing the composition to an object to prevent evaporation when the organic dye composition is scattered on the object.

In the present embodiment, the alcohol (B) is not particularly limited as long as it can adjust the viscosity in the organic dye composition the organic dye composition to 5 mPa·s or more and 45 mPa·s or less, but a biocompatible alcohol is preferable from the viewpoint of safety.

Preferably, the alcohol (B) of the present embodiment contains an alcohol having a boiling point of 180° C. or higher. Thereby, the contact angle of the organic dye composition of the present embodiment with respect to the above-described object can be 90° or less, and the wettability of the organic dye composition can be improved. When the boiling point is 180° C. or higher, it is considered that the volatility of the organic dye composition can be suppressed, and the composition can be suppressed from being quickly dried.

Preferred specific examples of the alcohol (B) include glycerin, propanediol, butanediol, pentanediol, hexanediol, heptanediol, butanetriol, pentanetriol, hexanetriol, and heptanetriol, and among them, glycerin and/or propanediol is preferably used from the viewpoint of safety. Furthermore, glycerin is preferably used from the viewpoint of more reliably detecting microorganisms and the like.

The content of the alcohol (B) is preferably 50% by mass or more with respect to the entire organic dye composition. Accordingly, it is considered that the viscosity, the contact angle, the wettability, and the like described above can be more reliably set to suitable ranges. The content of the alcohol (B) is more preferably 60% by mass or more, and further preferably 80% by mass or more. Also, the upper limit of the content is not particularly limited, but is preferably 90% by mass or less from the viewpoint of suppressing nozzle clogging at the time of spraying peculiar at the time of high viscosity.

### Water (C)

The organic dye composition of the present embodiment further contains water (C). The role of water (C) in the present embodiment is a biocompatible solvent.

As the water (C), normal purified water, distilled water, ion-exchanged water, sterilized water, or the like can be used.

The content of the water (C) in the organic dye composition of the present embodiment is preferably 5% by mass or more and 50% by mass or less with respect to the entire resin composition. Thereby, there is an advantage that the components in the organic dye composition are uniformly dissolved. A more preferable content is 5% by mass or more and 40% by mass or less.

### Others

The organic dye composition of the present embodiment may contain various additives and the like in addition to the components described above as long as the effects of the present invention are not impaired. In particular, an additive having no bactericidal action is preferable. This is because when the organic dye composition has a bactericidal action, there is a possibility that when the organic dye composition acts on microorganisms, the organic dye composition is inactivated or killed before viable cells of the microorganisms are sufficiently stained. Examples of the additive include binder resins, surfactants, pH adjusters, thickeners, ultraviolet absorbers, UV scattering agents, oxygen scavengers, antioxidants, fragrances, and solvents other than alcohol and water.

For example, when the organic dye composition of the present embodiment contains a binder resin, the binder resin functions to enhance fixability and wet spreading of droplets to an object to be sprayed when the organic dye composition is sprayed. The binder resin that can be used in the present embodiment is not particularly limited as long as it has no skin irritation and can be stably and uniformly dispersed in water and alcohol. From the viewpoint of skin irritation, it is preferable to select the binder resin from components listed in the component display name list of cosmetics based on the Pharmaceutical Affairs Law, and acrylic acid, methyl acrylate, ethyl acrylate, acrylic acid amide, n-propyl acrylate, n-butyl acrylate, isobutyl acrylate, octyl acrylate, 2-ethylhexyl acrylate, N,N-dimethylaminoethyl acrylate, acrylonitrile, methacrylic acid, ethyl methacrylate, methacrylic acid amide, n-propyl methacrylate, n-butyl methacrylate, isobutyl methacrylate, 2-ethylhexyl methacrylate, octyl methacrylate, hydroxyethyl methacrylate, N,N-dimethylaminoethyl methacrylate, and styrene, vinyl acetate, silicone macromers, fluorine-based monomers, alkoxysilane-unsaturated monomers and the like, which are known acrylic resins applied to cosmetics and the like, are preferable.

In addition, when the organic dye composition of the present embodiment contains a surfactant, the surfactant functions to uniformly disperse and dissolve the composition in the solution. The surfactant is also not particularly limited as long as it has no skin irritation and can be stably and uniformly dispersed in water and alcohol, but it is preferable to use a surfactant having no bactericidal action. This is because when the organic dye composition has a bactericidal action, there is a possibility that when the organic dye composition acts on microorganisms, the organic dye composition is inactivated or killed before viable cells of the microorganisms are sufficiently stained. Examples of the surfactant having no bactericidal action include anionic surfactants, amphoteric surfactants, and nonionic surfactants. Examples of the anionic surfactant include carboxylates, sulfonates, and sulfates. Examples of the amphoteric surfactant include carboxylates of an amino acid type or a betaine type. Examples of the nonionic surfactant include glycerin fatty acid esters, sorbitan fatty acid esters, sucrose fatty acid esters, polyoxyethylene alkyl ethers, polyoxyethylene alkylphenyl ethers, and polyoxyethylene polyoxypropylene glycols. Also, the HLB value of the surfactant used in the present embodiment is preferably 8 or more, and more preferably 8 to 19. When the HLB value of the surfactant is 8 or more, the surfactant is likely to be uniformly dispersed or dissolved in a higher alcohol, purified water, or the like. The HLB value is an index representing a ratio of relative affinity to both liquids in an oil-water system, and in general, those having a large HLB value have high affinity for water. The HLB value in the present specification is a value calculated by Griffin method.

### Preparation of Organic Dye Composition

The method for preparing the organic dye composition of the present embodiment is not particularly limited. For example, it can be prepared by adding the organic dye (A) and the alcohol (B) to a predetermined amount of water (C) and stirring the mixture.

### Applications of Organic Dye Composition

Since the organic dye composition of the present embodiment can visualize the presence or absence of microorganisms, it can be used for applications such as detection of microorganisms and confirmation of disinfection effects. Therefore, it is preferable to use the organic dye composition as a spray agent that can be sprayed and scattered in a wide range. By using as the spray agent, it is possible to quickly and easily detect microorganisms in an application object.

Specific examples of the object include kitchen sinks, cooking utensils, cooking tables, storage places of food, door knobs, wash basins, bathtubs, tiles, lines in food factories, shoes, floors, toilets, tables, products for infants (baby bottles, dishes, chairs, toys, and the like), other places touched by an unspecified number of people in public places, or fingers or bodies of humans. Therefore, the organic dye composition of the present embodiment preferably has a contact angle of 90° or less with respect to stainless steel, artificial marble, quartz, resin, enamel, wood, and the like constituting these objects. When the contact angle is 90° or less, the wettability of the organic dye composition to the object is enhanced. As a result, it is considered that the organic dye composition can remain in the detection object, and the detection of microorganisms can be performed more reliably. The contact angle is more preferably 80° or less, and further preferably 70° or less.

### Spray Agent

The spray agent of the present embodiment is not particularly limited as long as it contains the organic dye composition described above. For example, a spray agent is obtained by filling a spray container with the organic dye composition of the present embodiment.

As a container for containing the organic dye composition, a sealable 0.1 to 20 L light shielding container is preferable because it is convenient to carry. Examples of the material of the container include polyethylene terephthalate (PET), polypropylene (PP), polyethylene (PE), and glass. The organic dye contained in the organic dye composition of the present invention needs to be a light shielded container in order to prevent deterioration due to photolysis. Also, a colored light-shielding container may be used as the container, or the surface of the light transmitting container may be coated with a light shielding film or thin film (aluminum foil or the like).

In addition, the spray container is preferably a container with a trigger nozzle so as to facilitate spraying and scattering.

According to the spray agent of the present embodiment, the disinfection effect and the presence or absence of microorganisms in a wide range can be easily checked in a short time.

### Application Example

The organic dye composition and the spray agent of the present embodiment are useful in various applications. For example, the organic dye composition and the spray agent of the present embodiment can be incorporated into a system that visualizes the presence or absence of microorganisms, and such a visualization system can be applied to, for example, cleaning of a house.

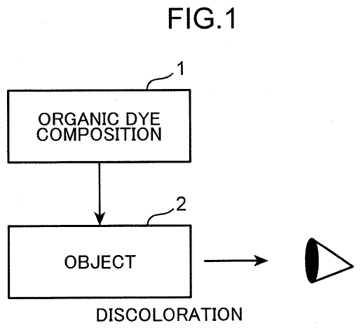

FIG. 1 is a block diagram showing a schematic configuration example of a microorganism visualization system (microorganism visualization method) using the organic dye composition according to the present embodiment. In the microorganism visualization system, first, an organic dye composition 1 according to the present embodiment is attached to an object 2 whose presence or absence of microorganisms is desired to be detected by spraying or the like. When a microorganism is present in the object, the organic dye composition 1 of the present embodiment reacts with the microorganism to produce a hydrolyzate, and discoloration occurs. By visually confirming the presence or absence of the discoloration, the presence or absence of microorganisms in the object can be easily examined.

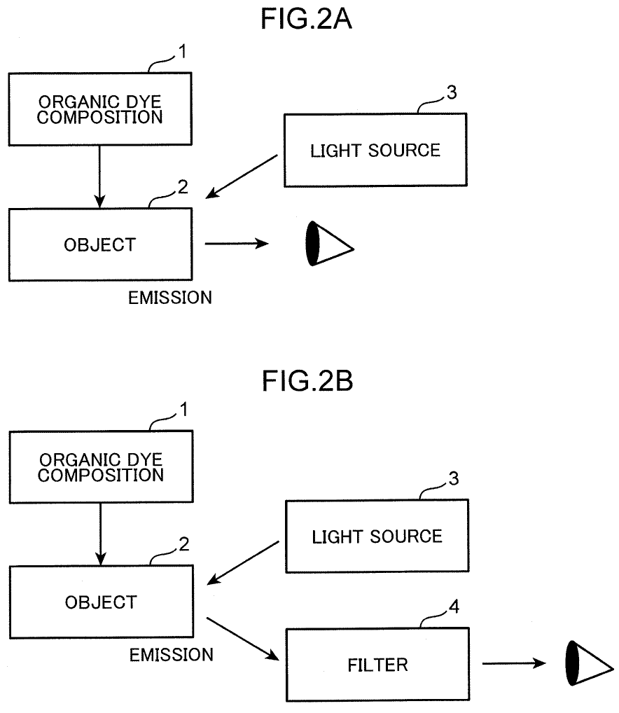

FIG. 2 (A) is a block diagram showing another configuration example of a microorganism visualization system (microorganism visualization method) using the organic dye composition according to the present embodiment. In this microorganism visualization system, the organic dye composition 1 according to the present embodiment is attached to the object 2 whose presence or absence of microorganisms is desired to be detected by spraying or the like, and then the object 2 is irradiated with light using a light source 3 that emits light having a wavelength of 400 nm or more and 550 nm or less. When the object contains microorganisms, the hydrolyzate of the organic dye (A) fluoresces by the light irradiation and the site attached is discolored, so that the site where the microorganisms are present can be visually recognized.

In addition, as shown in FIG. 2(B), the microorganism visualization system may further include a filter. In this case, in the microorganism visualization system, after the object 2 is irradiated with light from the light source 3, the site where the microorganisms are present is visually recognized through a filter 4. The filter 4 is preferably capable of filtering the wavelength of the light source 3. The visibility can be improved by cutting the reflected light or the like of the light source 3 using the filter 4. The filter 4 may also have a function of filtering ultraviolet light or blue light. This makes it possible to filter light having a wavelength harmful to the eyes and improve visibility while protecting the eyes.

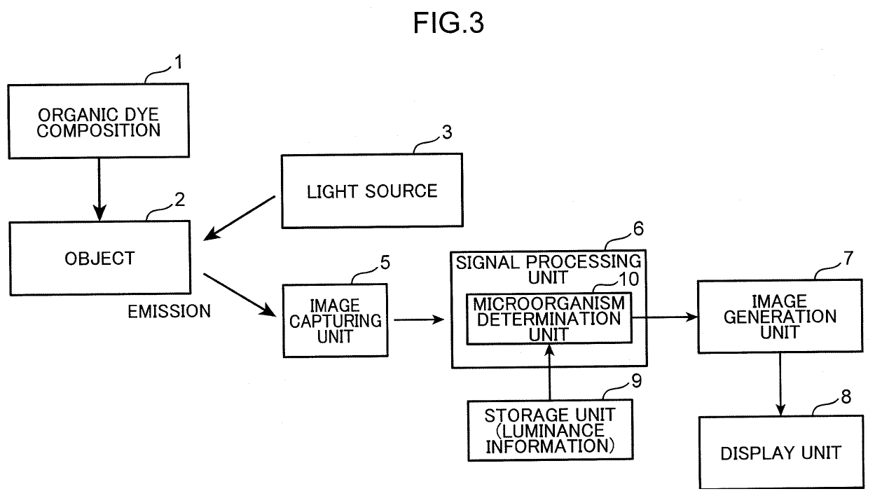

Furthermore, the microorganism visualization system may have a system configuration including a camera as in the block diagram shown in FIG. 3. By using a camera, the presence or absence of microorganisms can be confirmed by a captured image.

In that case, the system further includes an image capturing unit 5 that captures an image of the object 2; a signal processing unit 6 that processes a signal obtained from the image capturing unit 5; a storage unit 9 that holds luminance information on microorganisms; an image generation unit 7 that generates an image from the signal; and a display unit 8 that displays the generated image.

The signal processing unit 6 determines the presence or absence of microorganisms based on the luminance information obtained from the image capturing unit and the luminance information on microorganisms held in the storage unit (microorganism determination unit 10).

The image generation unit 7 generates an image based on the processing result of the signal processing unit 6. At this time, based on the microorganism determination information, an image in which a region determined that a microorganism is present is emphasized may be generated. Specifically, it is conceivable to change the color tone or contrast of the region determined that a microorganism is present to a color tone or contrast that is more easily visible.

By displaying the image thus obtained on the display unit 8, the site where the microorganisms are present can be easily identified.

In summary, the present embodiment also includes the following aspects (a microorganism visualization method and a microorganism visualization system), and the like.

### First Application Example

A microorganism visualization method including:
attaching, to an object, an organic dye composition containing:
an organic dye (A) that is discolored by hydrolysis and emits fluorescence by excitation light having a wavelength of 400 nm or more and 550 nm or less; an alcohol (B); and water (C),
in which a content rate of the organic dye (A) is 1 μmol/L or more, and
a viscosity is 5 mPa·s or more and 45 mPa·s or less; and
irradiating the object with light having a wavelength of 400 nm or more and 550 nm or less by a light source.

### Second Application Example

The microorganism visualization method according to the first application example, in which the object is observed through a filter after light irradiation.

### Third Application Example

A microorganism visualization system for attaching, to an object, an organic dye composition containing:
an organic dye (A) that is discolored by hydrolysis and emits fluorescence by excitation light having a wavelength of 400 nm or more and 550 nm or less; an alcohol (B); and water (C),
in which a content rate of the organic dye (A) is 1 μmol/L or more, and
a viscosity is 5 mPa·s or more and 45 mPa·s or less,
and visualizing microorganisms,
the microorganism visualization system including:
a light source that irradiates the object with light; an image capturing unit that captures an image of the object to obtain first luminance information; a storage unit that holds second luminance information on the microorganisms; a signal processing unit that compares the first luminance information with the second luminance information and determines the presence or absence of microorganisms; an image generation unit that generates an image from the signal; and a display unit that displays the generated image; and a microorganism visualization method using the microorganism visualization system.

This specification discloses techniques in various aspects as described above, and the main techniques among them are summarized below.

An organic dye composition according to a first aspect of the present invention contains an organic dye (A) that is discolored by hydrolysis and emits fluorescence by excitation light having a wavelength of 400 nm or more and 550 nm or less, an alcohol (B), and water (C), in which the content rate of the organic dye (A) is 1 μmol/L or more, and the viscosity is 5 mPa·s or more and 45 mPa·s or less.

An organic dye composition according to a second aspect is the organic dye composition of the first aspect, further, in which a contact angle with respect to at least one selected from stainless steel, artificial marble, quartz, resin, and enamel is 90° or less.

An organic dye composition according to a third aspect is the organic dye composition of the first and second aspects, in which the organic dye (A) is at least one selected from the compounds represented by Formulas (1) to (5).

An organic dye composition according to a fourth aspect is the organic dye composition according to any one of the first to third aspects, in which the alcohol (B) contains an alcohol having a boiling point of 180° C. or higher.

An organic dye composition according to a fifth aspect is the organic dye composition according to the fourth aspect, in which the alcohol is at least one selected from glycerin and propanediol.

An organic dye composition according to a sixth aspect is the organic dye compositions of the fourth and fifth aspects, in which a content of the alcohol (B) is 50% by mass or more with respect to an entire organic dye composition.

A spray agent according to a seventh aspect of the present invention contains the organic dye composition according to any one of the first to sixth aspects.

A microorganism visualization method according to an eighth aspect of the present invention includes attaching, to an object, the organic dye composition according to any one of the first to sixth aspects, and irradiating the object with light having a wavelength of 400 nm or more and 550 nm or less by a light source.

A microorganism visualization method according to a ninth aspect of the present invention is the microorganism visualization method according to the eighth aspect, further including observing the object through a filter after light irradiation.

A microorganism visualization system according to a tenth aspect of the present invention is a microorganism visualization system for attaching, to an object, the organic dye composition according to any one of the first to sixth aspects, and visualizing microorganisms, the microorganism visualization system including:
a light source that irradiates the object with light; an image capturing unit that captures an image of the object to obtain first luminance information; a storage unit that holds second luminance information on the microorganisms; a signal processing unit that compares the first luminance information with the second luminance information and determines the presence or absence of microorganisms; an image generation unit that generates an image from the signal; and a display unit that displays the generated image.

Hereinafter, the present invention will be described more specifically with reference to Examples, but the scope of the present invention is not limited thereto.

### EXAMPLES

### Test Example 1

Organic dye compositions of Examples 1-1 to 1-5 and Comparative Examples 1-1 to 1-6 were prepared by using a DMSO solution (excitation wavelength: 530 nm, dye stock solution concentration: 0.03 wt % (1 mmol/L in DMSO)) of an organic dye represented by Formula (4) as the organic dye (A), glycerin (boiling point: 290° C.) as the alcohol (B), and pure water obtained by a water production device “milliQ” manufactured by Merck Corporation as the water (C), and blending and stirring the respective components in the amounts (% by mass) shown in Table 1.

### Evaluation Method

### (1) Fluorescence Emission Upon Light Irradiation

100 μL of a bacterial liquid (E. coli, 106 cfu/mL) was added dropwise onto a slide glass. Then, a sample solution (organic dye composition) of each of Examples and Comparative Examples was placed in a spray bottle (φ39×147 mm, capacity 100 ml), and sprayed (one push) to the bacterial liquid on the slide glass. Thereafter, after leaving for 15 minutes, the following fluorescence observation and color change observation were performed.
Fluorescence observation (visual visibility)

For fluorescence observation, the sprayed part was irradiated with an LED light (530 nm output, color: green), and visual observation was performed through a red filter. The evaluation criteria were as follows.
good: Emission of the solution can be clearly visually recognized
fair: Emission of the solution can be visually recognized but very weak
poor: Emission of the solution cannot be visually recognized
Color change
Color change was visually observed and evaluated according to the following criteria:
good: Color change of the solution can be clearly visually recognized
fair: Color change of the solution can be visually recognized but very weak
poor: Color change of the solution cannot be visually recognized or no color change occurs

### (2) Viscosity

The viscosity (mPa·s) of the sample solution (organic dye composition) of each of Examples and Comparative Examples was measured with a rheometer (cone-plate viscometer). The measurement conditions were a shear rate of 1,000 [1/s] at 25° C.

### (3) Contact Angle

100 μL of the sample solution (organic dye composition) of each of Examples and Comparative Examples was dropped onto a substrate made of polyethylene, polypropylene, and stainless steel. The contact angle of a droplet on the substrate was measured (the image was captured with a camera from horizontal surface, and the contact angle was measured) by a droplet method.

### (4) Droplet Fixation to Object

The sample solution (organic dye composition) was placed in a spray bottle (39×147 mm, capacity 100 ml), and sprayed (one push) on a substrate made of polyethylene, polypropylene, and stainless steel. Thereafter, evaluation was performed according to the following criteria by visual observation. In each of Examples and Comparative Examples, since there was no limit in the evaluation result depending on the material of the substrate, the results are collectively described.
good: The contact angle with respect to the substrate to be sprayed was 90° or less, and when spraying the solution, the solution could be fixed without much scattering outside the spray region
poor: The contact angle was larger than 90° with respect to the substrate to be sprayed, and when spraying the solution, splashing and rolling of the droplets from the spray target were large, and the droplets scattered outside the spray region

### (5) Dye Stability

It was confirmed whether the organic dye (A) was spontaneously decomposed (hydrolyzed) in the organic dye composition, and stability (durability) of the dye was confirmed. First, 1 mL of the sample solution (organic dye composition) of each of Examples and Comparative Examples was sealed in a colorless and transparent microtube. Then, after leaving for 2 hours, the solution part was irradiated with an LED light (530 nm output, color: green), and visual observation was performed through a red filter to confirm whether or not fluorescence was emitted at a visible level. The evaluation criteria were as follows.
good: The organic dye was not decomposed in the solution and was stable for 2 hours or more (fluorescence could not be visually recognized after 2 hours)
poor: The organic dye was decomposed over time in the solution and fluorescence was visible after 2 hours

The above results are summarized in Table 1.

TABLE 1

| | | | Compar- | Compar- | | | | Compar- | Compar- |
|---|---|---|---|---|---|---|---|---|---|
| | | | ative | ative | Exam | Exam- | Exam- | ative | ative |
| | | | Exam- | Exam- | ple | ple | ple | Exam- | Exam- |
| | | | ple 1-1 | ple 1-2 | 1-1 | 1-2 | 1-3 | ple 1-3 | ple 1-4 |
| Microorganism | E. coli (10⁶ cfu/mL) | | | | | | | | |
| Organic dye | Organic dye (A) | Concentration (μmol/L) in | 0.01 | 0.1 | 1 | 10 | 20 | 20 | 20 |
| composition | (Dye of | total organio dye composition | | | | | | | |
| | Formula (4)) | Content (% by mass (of dye solution)) | 2 | 2 | 2 | 2 | 2 | 2 | 2 |
| | | (A)/{(A) + (B) + (C)} × 100 | | | | | | | |
| | Alcohol (B): | Content (% by mass) | 60 | 60 | 60 | 60 | 60 | 0 | 20 |
| | Glycerin | (B)/{(A) + (B) + (C)} × 100 | | | | | | | |
| | Water (C) | Content (% by mass) | 38 | 38 | 38 | 38 | 38 | 98 | 78 |
| | | (C)/{(A) + (B) + (C)} × 100 | | | | | | | |
| Fluorescence/ | Fluorescence emission upon light | | poor | poor | good | good | good | good | good |
| color change | irradiation (visual visibility) | | | | | | | | |
| upon dye | Color change | | poor | poor | fair | good | good | good | good |
| hydrolysis | | | | | | | | | |
| Physical | Viscosity (mPa · s) | | 8.7 | 8.7 | 8.7 | 8.7 | 8.7 | 1 | 1.4 |
| properties | Contact angle | vs. Polyethylene | 60 | 60 | 60 | 60 | 60 | 90 | 70 |
| | (degrees) | vs. Polypropylene | 60 | 60 | 60 | 60 | 60 | 90 | 80 |
| | | vs. Stainless steel | 60 | 60 | 60 | 60 | 60 | 90 | 90 |
| Evaluation as | Droplet fixation to object | | good | good | good | good | good | poor | good |
| spray agent, | Dye stability | | good | good | good | good | good | poor | poor |
| etc. | | | | | | | | | |

| | | | Compar- | | | Compar- |
|---|---|---|---|---|---|---|
| | | | ative | Exam- | Exam- | ative |
| | | | Exam- | ple | ple | Exam- |
| | | | ple 1-5 | 1-4 | 1-5 | ple 1-6 |
| Microorganism | E. coli (10⁶ cfu/mL) | | | | | |
| Organic dye | Organic dye (A) | Concentration (μmol/L) in | 20 | 20 | 20 | 20 |
| composition | (Dye of | total organio dye composition | | | | |
| | Formula (4)) | Content (% by mass (of dye solution)) | 2 | 2 | 2 | 2 |
| | | (A)/{(A) + (B) + (C)} × 100 | | | | |
| | Alcohol (B): | Content (% by mass) | 40 | 80 | 90 | 98 |
| | Glycerin | (B)/{(A) + (B) + (C)} × 100 | | | | |
| | Water (C) | Content (% by mass) | 58 | 18 | 8 | 0 |
| | | (C)/{(A) + (B) + (C)} × 100 | | | | |
| Fluorescence/ | Fluorescence emission upon light | | good | good | good | good |
| color change | irradiation (visual visibility) | | | | | |
| upon dye | Color change | | good | good | good | good |
| hydrolysis | | | | | | |
| Physical | Viscosity (mPa · s) | | 2.9 | 39 | 42 | 67 |
| properties | Contact angle | vs. Polyethylene | 60 | 70 | 60 | 50 |
| | (degrees) | vs. Polypropylene | 70 | 80 | 40 | 50 |
| | | vs. Stainless steel | 80 | 70 | 50 | 40 |
| Evaluation as | Droplet fixation to object | | good | good | good | Not sprayable due |
| spray agent, | | | | | | to high viscosity |

| | | | | | |
|---|---|---|---|---|---|
| etc. | Dye stability | poor | good | good | good |

### Test Example 2

### Preparation of Organic Dye Composition

Organic dye compositions of Examples 2-1 to 2-5 and Comparative Examples 2-1 to 2-6 were prepared by using a DMSO solution (excitation wavelength: 490 nm, dye stock solution concentration: 0.1 wt % (1 mg/mL in DMSO)) of an organic dye represented by Formula (2) as the organic dye (A), glycerin (boiling point: 290° C.) as the alcohol (B), and pure water obtained by a water production device “milliQ” manufactured by Merck Corporation as the water (C), and blending and stirring the respective components in the amounts (% by mass) shown in Table 2.

Using each of the obtained organic dye compositions, the evaluation tests (1) to (5) were performed in the same manner as in Test Example 1. The results are summarized in Table 2.

TABLE 2

| | | | Compar- | Compar- | | | | Compar- |
|---|---|---|---|---|---|---|---|---|
| | | | ative | ative | Exam- | Exam- | Exam- | ative |
| | | | Exam- | Exam- | ple | ple | ple | Exam- |
| | | | ple 2-1 | ple 2-2 | 2-1 | 2-2 | 2-3 | ple 2-3 |
| Microorganism | E. coli (10⁶ cfu/mL) | | | | | | | |
| Organic dye | Organic dye (A) | Concentration (μmol/L) in | 0.01 | 0.1 | 1 | 10 | 20 | 20 |
| composition | (Dye of | total organic dye composition | | | | | | |
| | Formula (2)) | Content (% by mass (of dye solution)) | 2 | 2 | 2 | 2 | 2 | 2 |
| | | (A)/{(A) + (B) + (C)} × 100 | | | | | | |
| | Alcohol (B): | Content (% by mass) | 60 | 60 | 60 | 60 | 60 | 0 |
| | Glycerin | (B)/{(A) + (B) + (C)} × 100 | | | | | | |
| | Water (C) | Content (% by mass) | 38 | 38 | 38 | 38 | 38 | 98 |
| | | (C)/{(A) + (B) + (C)} × 100 | | | | | | |
| Fluorescence/ | Fluorescence emission upon light | | poor | poor | good | good | good | good |
| color change | irradiation (visual visibility) | | | | | | | |
| upon dye | Color change | | poor | poor | fair | fair | fair | fair |
| hydrolysis | | | | | | | | |
| Physical | Viscosity (mPa · s) | | 8.7 | 8.7 | 8.7 | 8.7 | 8.7 | 1 |
| properties | Contact angle | vs. Polyethylene | 60 | 60 | 60 | 60 | 60 | 90 |
| | (degrees) | vs. Polypropylene | 60 | 60 | 60 | 60 | 60 | 90 |
| | | vs. Stainless steel | 60 | 60 | 60 | 60 | 60 | 90 |
| Evaluation as | Droplet fixation to object | | good | good | good | good | good | poor |
| spray agent, | Dye stability | | good | good | good | good | good | poor |
| etc. | | | | | | | | |

| | | | Compar- | Compar- | | | Compar- |
|---|---|---|---|---|---|---|---|
| | | | ative | ative | Exam- | Exam- | ative |
| | | | Exam- | Exam- | ple | ple | Exam- |
| | | | ple 2-4 | ple 2-5 | 2-4 | 2-5 | ple 2-6 |
| Microorganism | E. coli (10⁶ cfu/mL) | | | | | | |
| Organic dye | Organic dye (A) | Concentration (μmol/L) in | 20 | 20 | 20 | 20 | 20 |
| composition | (Dye of | total organic dye composition | | | | | |
| | Formula (2)) | Content (% by mass (of dye solution)) | 2 | 2 | 2 | 2 | 2 |
| | | (A)/{(A) + (B) + (C)} × 100 | | | | | |
| | Alcohol (B): | Content (% by mass) | 20 | 40 | 80 | 90 | 98 |
| | Glycerin | (B)/{(A) + (B) + (C)} × 100 | | | | | |
| | Water (C) | Content (% by mass) | 78 | 58 | 18 | 8 | 0 |
| | | (C)/{(A) + (B) + (C)} × 100 | | | | | |
| Fluorescence/ | Fluorescence emission upon light | | good | good | good | good | good |
| color change | irradiation (visual visibility) | | | | | | |
| upon dye | Color change | | fair | fair | fair | fair | fair |
| hydrolysis | | | | | | | |
| Physical | Viscosity (mPa · s) | | 1.4 | 2.9 | 39 | 42 | 67 |
| properties | Contact angle | vs. Polyethylene | 70 | 60 | 70 | 60 | 50 |
| | (degrees) | vs. Polypropylene | 80 | 70 | 80 | 40 | 50 |
| | | vs. Stainless steel | 90 | 80 | 70 | 50 | 40 |
| Evaluation as | Droplet fixation to object | | good | good | good | good | Not sprayable |
| spray agent, | | | | | | | due to high |
| etc. | | | | | | | viscosity |

| | | | | | |
|---|---|---|---|---|---|
| Dye stability | poor | poor | good | good | good |

### Test Example 3

### Preparation of Organic Dye Composition

Organic dye compositions of Examples 3-1 to 3-5 and Comparative Examples 3-1 to 3-6 were prepared by using a DMSO solution (excitation wavelength: 530 nm, dye stock solution concentration: 0.03 wt % (1 mmol/L in DMSO)) of an organic dye represented by Formula (4) as the organic dye (A), glycerin (boiling point: 290° C.) as the alcohol (B), and pure water obtained by a water production device “milliQ” manufactured by Merck Corporation as the water (C), and blending and stirring the respective components in the amounts (% by mass) shown in Table 3.

Each of the obtained organic dye compositions was used, and the evaluation tests (1) to (5) were performed in the same manner as in Test Example 1 except that Staphylococcus aureus (10⁵ cfu/mL) was used as the bacterial liquid. The results are summarized in Table 3.

TABLE 3

| | | | Compar- | Compar- | | | | Compar- | Compar- |
|---|---|---|---|---|---|---|---|---|---|
| | | | ative | ative | Exam- | Exam- | Exam- | ative | ative |
| | | | Exam- | Exam- | ple | ple | ple | Exam- | Exam- |
| | | | ple 3-1 | ple 3-2 | 3-1 | 3-2 | 3-3 | ple 3-3 | ple 3-4 |
| Microorganism | Staphylococcus aureus (10⁵ cfu/mL) | | | | | | | | |
| Organic dye | Organic dye | Concentration (μmol/L) in | 0.01 | 0.1 | 1 | 10 | 20 | 20 | 20 |
| composition | (A) (Dye of | total organic dye composition | | | | | | | |
| | Formula | Content (% by mass (of dye solution)) | 2 | 2 | 2 | 2 | 2 | 2 | 2 |
| | (4)) | (A)/{(A) + (B) + (C)} × 100 | | | | | | | |
| | Alcohol (B): | Content (% by mass) | 60 | 60 | 60 | 60 | 60 | 0 | 20 |
| | Glycerin | (B)/{(A) + (B) + (C)} × 100 | | | | | | | |
| | Water (C) | Content (% by mass) | 38 | 38 | 38 | 38 | 38 | 98 | 78 |
| | | (C)/{(A) + (B) + (C)} × 100 | | | | | | | |
| Fluorescence/ | Fluorescence emission upon light | | poor | poor | good | good | good | good | good |
| color change | irradiation (visual visibility) | | | | | | | | |
| upon dye | Color change | | poor | poor | fair | good | good | good | good |
| hydrolysis | | | | | | | | | |
| Physical | Viscosity (mPa · s) | | 8.7 | 8.7 | 8.7 | 8.7 | 8.7 | 1 | 1.4 |
| properties | Contact | vs. Polyethylene | 60 | 60 | 60 | 60 | 60 | 90 | 70 |
| | angle | vs. Polypropylene | 60 | 60 | 60 | 60 | 60 | 90 | 80 |
| | (degrees) | vs. Stainless steel | 60 | 60 | 60 | 60 | 60 | 90 | 90 |
| Evaluation | Droplet fixation to object | | good | good | good | good | good | Poor | good |
| as spray | Dye stability | | good | good | good | good | good | poor | poor |
| agent, | | | | | | | | | |
| etc. | | | | | | | | | |

| | | | Compar- | | | Compar- |
|---|---|---|---|---|---|---|
| | | | ative | Exam- | Exam- | ative |
| | | | Exam- | ple | ple | Exam- |
| | | | ple 3-5 | 3-4 | 3-5 | ple 3-6 |
| Microorganism | Staphylococcus aureus (10⁵ cfu/mL) | | | | | |
| Organic dye | Organic dye | Concentration (μmol/L) in | 20 | 20 | 20 | 20 |
| composition | (A) (Dye of | total organic dye composition | | | | |
| | Formula | Content (% by mass (of dye solution)) | 2 | 2 | 2 | 2 |
| | (4)) | (A)/{(A) + (B) + (C)} × 100 | | | | |
| | Alcohol (B): | Content (% by mass) | 40 | 80 | 90 | 98 |
| | Glycerin | (B)/{(A) + (B) + (C)} × 100 | | | | |
| | Water (C) | Content (% by mass) | 58 | 18 | 8 | 0 |
| | | (C)/{(A) + (B) + (C)} × 100 | | | | |
| Fluorescence/ | Fluorescence emission upon light | | good | good | good | good |
| color change | irradiation (visual visibility) | | | | | |
| upon dye | Color change | | good | good | good | good |
| hydrolysis | | | | | | |
| Physical | Viscosity (mPa · s) | | 2.9 | 39 | 42 | 67 |
| properties | Contact | vs. Polyethylene | 60 | 70 | 60 | 50 |
| | angle | vs. Polypropylene | 70 | 80 | 40 | 50 |
| | (degrees) | vs. Stainless steel | 80 | 70 | 50 | 40 |
| Evaluation | Droplet fixation to object | | good | good | good | Not |
| as spray | | | | | | sprayable |
| agent, | | | | | | due to high |
| etc. | | | | | | viscosity |

| | | | | |
|---|---|---|---|---|
| Dye stability | poor | good | good | good |

### Test Example 4

### Preparation of Organic Dye Composition

Organic dye compositions of Examples 4-1 to 4-5 and Comparative Examples 4-1 to 4-6 were prepared by using a DMSO solution (excitation wavelength: 490 nm, dye stock solution concentration: 0.1 wt % (1 mg/mL in DMSO)) of an organic dye represented by Formula (2) as the organic dye (A), glycerin (boiling point: 290° C.) as the alcohol (B), and pure water obtained by a water production device “milliQ” manufactured by Merck Corporation as the water (C), and blending and stirring the respective components in the amounts (% by mass) shown in Table 4.

Each of the obtained organic dye compositions was used, and the evaluation tests (1) to (5) were performed in the same manner as in Test Example 1 except that Staphylococcus aureus (10⁵ cfu/mL) was used as the bacterial liquid. The results are summarized in Table 4.

TABLE 4

| | | | Compar- | Compar- | | | | Compar- | Compar- |
|---|---|---|---|---|---|---|---|---|---|
| | | | ative | ative | Exam- | Exam- | Exam- | ative | ative |
| | | | Exam- | Exam- | ple | ple | ple | Exam- | Exam- |
| | | | ple 4-1 | ple 4-2 | 4-1 | 4-2 | 4-3 | ple 4-3 | ple 4-4 |
| Microorganism | Staphylococcus aureus (10⁵ cfu/mL) | | | | | | | | |
| Organic dye | Organic dye | Concentration (μmol/L) in | 0.01 | 0.1 | 1 | 10 | 20 | 20 | 20 |
| composition | (A) (Dye of | total organic dye composition | | | | | | | |
| | Formula | Content (% by mass (of dye solution)) | 2 | 2 | 2 | 2 | 2 | 2 | 2 |
| | (2)) | (A)/{A) + (B) + (C)} × 100 | | | | | | | |
| | Alcohol (B): | Content (% by mass) | 60 | 60 | 60 | 60 | 60 | 0 | 20 |
| | Glycerin | (B)/{(A) + (B) + (C)} × 100 | | | | | | | |
| | Water (C) | Content (% by mass) | 38 | 38 | 38 | 38 | 38 | 98 | 78 |
| | | (C)/{(A) + (B) + (C)} × 100 | | | | | | | |
| Fluorescence/ | Fluorescence emission upon light | | poor | poor | good | good | good | good | good |
| color change | irradiation (visual visibility) | | | | | | | | |
| upon dye | Color change | | poor | poor | fair | fair | fair | fair | fair |
| hydrolysis | | | | | | | | | |
| Physical | Viscosity (mPa · s) | | 8.7 | 8.7 | 8.7 | 8.7 | 3.7 | 1 | 1.4 |
| properties | Contact | vs. Polyethylene | 60 | 60 | 60 | 60 | 60 | 90 | 70 |
| | angle | vs. Polypropylene | 60 | 60 | 60 | 60 | 60 | 90 | 80 |
| | (degrees) | vs. Stainless steel | 60 | 60 | 60 | 60 | 60 | 90 | 90 |
| Evaluation | Droplet fixation to object | | good | good | good | good | good | poor | good |
| as spray | Dye stability | | good | good | good | good | good | poor | poor |
| agent, etc. | | | | | | | | | |

| | | | Compar- | | | Compar- |
|---|---|---|---|---|---|---|
| | | | ative | Exam- | Exam- | ative |
| | | | Exam- | ple | ple | Exam- |
| | | | ple 4-5 | 4-4 | 4-5 | ple 4-6 |
| Microorganism | Staphylococcus aureus (10⁵ cfu/mL) | | | | | |
| Organic dye | Organic dye | Concentration (μmol/L) in | 20 | 20 | 20 | 20 |
| composition | (A) (Dye of | total organic dye composition | | | | |
| | Formula | Content (% by mass (of dye solution)) | 2 | 2 | 2 | 2 |
| | (2)) | (A)/{(A) + (B) + (C)] × 100 | | | | |
| | Alcohol (B): | Content (% by mass) | 40 | 80 | 90 | 98 |
| | Glycerin | (B)/{(A) + (B) + (C)] × 100 | | | | |
| | Water (C) | Content (% by mass) | 58 | 18 | 8 | 0 |
| | | (C)/{(A) + (B) + (C)] × 100 | | | | |
| Fluorescence/ | Fluorescence emission upon light | | good | good | good | good |
| color change | irradiation (visual visibility) | | | | | |
| upon dye | Color change | | fair | fair | fair | fair |
| hydrolysis | | | | | | |
| Physical | Viscosity (mPa · s) | | 2.9 | 39 | 42 | 67 |
| properties | Contact | vs. Polyethylene | 60 | 70 | 60 | 50 |
| | angle | vs. Polypropylene | 70 | 80 | 40 | 50 |
| | (degrees) | vs. Stainless steel | 80 | 70 | 50 | 40 |
| Evaluation | Droplet fixation to object | | good | good | good | Not |
| as spray | | | | | | sprayable |
| agent, etc. | | | | | | due to high |
| | | | | | | viscosity |

| | | | | |
|---|---|---|---|---|
| Dye stability | poor | good | good | good |

### Test Example 5

### Preparation of Organic Dye Composition

Organic dye compositions of Examples 5-1 to 5-6 and Comparative Examples 5-1 to 5-5 were prepared by using a DMSO solution (excitation wavelength: 530 nm, dye stock solution concentration: 0.03 wt % (1 mmol/L in DMSO)) of an organic dye represented by Formula (4) as the organic dye (A), 1,3-propanediol (boiling point: 213° C.) as the alcohol (B), and pure water obtained by a water production device “milliQ” manufactured by Merck Corporation as the water (C), and blending and stirring the respective components in the amounts (% by mass) shown in Table 5.

Using each of the obtained organic dye compositions, the evaluation tests (1) to (5) were performed in the same manner as in Test Example 1. The results are summarized in Table 5.

TABLE 5

| | | | Compar- | Compar- | | | | Compar- |
|---|---|---|---|---|---|---|---|---|
| | | | ative | ative | Exam- | Exam- | Exam- | ative |
| | | | Exam- | Exam- | ple | ple | ple | Exam- |
| | | | ple 5-1 | ple 5-2 | 5-1 | 5-2 | 5-3 | ple 5-3 |
| Microorganism | E. coli (10⁶ cfu/mL) | | | | | | | |
| Organic dye | Organic dye | Concentration (μmol/L) in | 0.01 | 0.1 | 1 | 10 | 20 | 20 |
| composition | (A) | total organic dye composition | | | | | | |
| | (Dye of Formula | Content (% by mass (of dye solution)) | 2 | 2 | 2 | 2 | 2 | 2 |
| | (4)) | (A)/{(A) + (B) + (C)} × 100 | | | | | | |
| | Alcohol (B): | Content (% by mass) | 60 | 60 | 60 | 60 | 60 | 0 |
| | Propanediol | (B)/{(A) + (B) + (C)} × 100 | | | | | | |
| | Water (C) | Content (% by mass) | 38 | 38 | 38 | 38 | 38 | 98 |
| | | (C)/{(A) + (B) + (C)} × 100 | | | | | | |
| Fluorescence/ | Fluorescence emission upon light | | poor | poor | fair | fair | fair | fair |
| color change | irradiation (visual visibility) | | | | | | | |
| upon dye | Color change | | poor | poor | fair | fair | fair | fair |
| hydrolysis | | | | | | | | |
| Physical | Viscosity (mPa · s) | | 8 | 8 | 8 | 8 | 8 | 1 |
| properties | Contact | vs. Polyethylene | 50 | 50 | 50 | 50 | 50 | 90 |
| | angle | vs. Polypropylene | 40 | 40 | 40 | 40 | 40 | 90 |
| | (degrees) | vs. Stainless steel | 40 | 40 | 40 | 40 | 40 | 90 |

| | | | | | | | |
|---|---|---|---|---|---|---|---|
| Evaluation | Droplet fixation to object | good | good | good | good | good | poor |
| as spray | Dye stability | good | good | good | good | good | poor |
| agent, etc. | | | | | | | |

| | | | Compar- | Compar- | | | |
|---|---|---|---|---|---|---|---|
| | | | ative | ative | Exam- | Exam- | Exam- |
| | | | Exam- | Exam- | ple | ple | ple |
| | | | ple 5-4 | ple 5-5 | 5-4 | 5-5 | 5-6 |
| Microorganism | E. coli (10⁶ cfu/mL) | | | | | | |
| Organic dye | Organic dye | Concentration (μmol/L) in | 20 | 20 | 20 | 20 | 20 |
| composition | (A) | total organic dye composition | | | | | |
| | (Dye of Formula | Content (% by mass (of dye solution)) | 2 | 2 | 2 | 2 | 2 |
| | (4)) | (A)/{(A) + (B) + (C)} × 100 | | | | | |
| | Alcohol (B): | Content (% by mass) | 20 | 40 | 80 | 90 | 98 |
| | Propanediol | (B)/{(A) + (B) + (C)} × 100 | | | | | |
| | Water (C) | Content (% by mass) | 78 | 58 | 18 | 8 | 0 |
| | | (C)/{(A) + (B) + (C)} × 100 | | | | | |
| Fluorescence/ | Fluorescence emission upon light | | fair | fair | fair | fair | fair |
| color change | irradiation (visual visibility) | | | | | | |
| upon dye | Color change | | fair | fair | fair | fair | fair |
| hydrolysis | | | | | | | |
| Physical | Viscosity (mPa · s) | | 2 | 3.6 | 15 | 23 | 33 |
| properties | Contact | vs. Polyethylene | 60 | 60 | 50 | 50 | 50 |
| | angle | vs. Polypropylene | 60 | 40 | 40 | 40 | 40 |
| | (degrees) | vs. Stainless steel | 80 | 60 | 40 | 30 | 30 |

| | | | | | | |
|---|---|---|---|---|---|---|
| Evaluation | Droplet fixation to object | poor | poor | good | good | good |
| as spray | Dye stability | poor | poor | good | good | good |
| agent, etc. | | | | | | |

### Test Example 6

### Preparation of Organic Dye Composition

Organic dye compositions of Examples 6-1 to 6-6 and Comparative Examples 6-1 to 6-5 were prepared by using a DMSO solution (excitation wavelength: 490 nm, dye stock solution concentration: 0.1 wt % (1 mg/mL in DMSO)) of an organic dye represented by Formula (2) as the organic dye (A), 1,3-propanediol (boiling point: 213° C.) as the alcohol (B), and pure water obtained by a water production device “milliQ” manufactured by Merck Corporation as the water (C), and blending and stirring the respective components in the amounts (% by mass) shown in Table 6.

Using each of the obtained organic dye compositions, the evaluation tests (1) to (5) were performed in the same manner as in Test Example 1. The results are summarized in Table 6.

TABLE 6

| | | | Compar- | Compar- | | | | Compar- | Compar- |
|---|---|---|---|---|---|---|---|---|---|
| | | | ative | ative | Exam- | Exam- | Exam- | ative | ative |
| | | | Exam- | Exam- | ple | ple | ple | Exam- | Exam- |
| | | | ple 6-1 | ple 6-2 | 6-1 | 6-2 | 6-3 | ple 6-3 | ple 6-4 |
| Microorganism | E. coli (10⁶ cfu/mL) | | | | | | | | |
| Organic dye | Organic dye | Concentration (μmol/L) in | 0.01 | 0.1 | 1 | 10 | 20 | 20 | 20 |
| composition | (A) (Dye of | total organic dye composition | | | | | | | |
| | Formula (2)) | Content (% by mass (of dye solution)) | 2 | 2 | 2 | 2 | 2 | 2 | 2 |
| | | (A)/{(A) + (B) + (C)} × 100 | | | | | | | |
| | Alcohol (B): | Content (% by mass) | 60 | 60 | 60 | 60 | 60 | 0 | 20 |
| | Propanediol | (B)/{(A) + (B) + (C)} × 100 | | | | | | | |
| | Water (C) | Content (% by mass) | 38 | 38 | 38 | 38 | 38 | 98 | 78 |
| | | (C)/{(A) + (B) + (C)} × 100 | | | | | | | |
| Fluorescence/ | Fluorescence emission upon light | | poor | poor | fair | fair | fair | fair | fair |
| color change | irradiation (visual visibility) | | | | | | | | |
| upon dye | Color change | | poor | poor | fair | fair | fair | fair | fair |
| hydrolysis | | | | | | | | | |
| Physical | Viscosity (mPa · s) | | 8 | 8 | 8 | 8 | 8 | 1 | 2 |
| properties | Contact | vs. Polyethylene | 50 | 50 | 50 | 50 | 50 | 90 | 60 |
| | angle | vs. Polypropylene | 40 | 40 | 40 | 40 | 40 | 90 | 60 |
| | (degrees) | vs. Stainless steel | 40 | 40 | 40 | 40 | 40 | 90 | 80 |

| | | | | | | | | |
|---|---|---|---|---|---|---|---|---|
| Evaluation | Droplet fixation to object | good | good | good | good | good | poor | poor |
| as spray | Dye stability | good | good | good | good | good | poor | poor |
| agent, etc. | | | | | | | | |

| | | | Compar- | | | |
|---|---|---|---|---|---|---|
| | | | ative | Exam- | Exam- | Exam- |
| | | | Exam- | ple | ple | ple |
| | | | ple 6-5 | 6-4 | 6-5 | 6-6 |
| Microorganism | E. coli (10⁶ cfu/mL) | | | | | |
| Organic dye | Organic dye | Concentration (μmol/L) in | 20 | 20 | 20 | 20 |
| composition | (A) (Dye of | total organic dye composition | | | | |
| | Formula (2)) | Content (% by mass (of dye solution)) | 2 | 2 | 2 | 2 |
| | | (A)/{(A) + (B) + (C)} × 100 | | | | |
| | Alcohol (B): | Content (% by mass) | 40 | 80 | 90 | 98 |
| | Propanediol | (B)/{(A) + (B) + (C)} × 100 | | | | |
| | Water (C) | Content (% by mass) | 58 | 18 | 8 | 0 |
| | | (C)/{(A) + (B) + (C)} × 100 | | | | |
| Fluorescence/ | Fluorescence emission upon light | | fair | fair | fair | fair |
| color change | irradiation (visual visibility) | | | | | |
| upon dye | Color change | | fair | fair | fair | fair |
| hydrolysis | | | | | | |
| Physical | Viscosity (mPa · s) | | 3.6 | 15 | 23 | 33 |
| properties | Contact | vs. Polyethylene | 60 | 50 | 50 | 50 |
| | angle | vs. Polypropylene | 40 | 40 | 40 | 40 |
| | (degrees) | vs. Stainless steel | 60 | 40 | 30 | 30 |

| | | | | | |
|---|---|---|---|---|---|
| Evaluation | Droplet fixation to object | poor | good | good | good |
| as spray | Dye stability | poor | good | good | good |
| agent, etc. | | | | | |

### Test Example 7

### Preparation of Organic Dye Composition

Organic dye compositions of Examples 7-1 to 7-6 and Comparative Examples 7-1 to 7-5 were prepared by using a DMSO solution (excitation wavelength: 530 nm, dye stock solution concentration: 0.03 wt % (1 mmol/L in DMSO)) of an organic dye represented by Formula (4) as the organic dye (A), 1,3-propanediol (boiling point: 213° C.) as the alcohol (B), and pure water obtained by a water production device “milliQ” manufactured by Merck Corporation as the water (C), and blending and stirring the respective components in the amounts (% by mass) shown in Table 7.

Each of the obtained organic dye compositions was used, and the evaluation tests (1) to (5) were performed in the same manner as in Test Example 1 except that Staphylococcus aureus (105 cfu/mL) was used as the bacterial liquid. The results are summarized in Table 7.

TABLE 7

| | | | Compar- | Compar- | | | | Compar- | Compar- |
|---|---|---|---|---|---|---|---|---|---|
| | | | ative | ative | Exam- | Exam- | Exam- | ative | ative |
| | | | Exam- | Exam- | ple | ple | ple | Exam- | Exam- |
| | | | ple 7-1 | ple 7-2 | 7-1 | 7-2 | 7-3 | ple 7-3 | ple 7-4 |
| Microorganism | Staphylococcus aureus (10⁵ cfu/mL) | | | | | | | | |
| Organic dye | Organic dye | Concentration (μmol/L) in | 0.01 | 0.1 | 1 | 10 | 20 | 20 | 20 |
| composition | (A) (Dye of | total organic dye composition | | | | | | | |
| | Formula (4)) | Content (% by mass (of dye solution) | 2 | 2 | 2 | 2 | 2 | 2 | 2 |
| | | (A)/{(A) + (B) + (C)} × 100 | | | | | | | |
| | Alcohol (B): | Content (% by mass) | 60 | 60 | 60 | 60 | 60 | 0 | 20 |
| | Propanediol | (B)/{(A) + (B) + (C)} × 100 | | | | | | | |
| | Water (C) | Content (% by mass) | 38 | 38 | 38 | 38 | 38 | 98 | 78 |
| | | (C)/{(A) + (B) + (C)} × 100 | | | | | | | |
| Fluorescence/ | Fluorescence emission upon light | | poor | poor | fair | fair | fair | fair | fair |
| color change | irradiation (visual visibility) | | | | | | | | |
| upon dye | Color change | | poor | poor | fair | fair | fair | fair | fair |
| hydrolysis | | | | | | | | | |
| Physical | Viscosity (mPa · s) | | 8 | 8 | 8 | 8 | 8 | 1 | 2 |
| properties | Contact | vs. Polyethylene | 50 | 50 | 50 | 50 | 50 | 90 | 60 |
| | angle | vs. Polypropylene | 40 | 40 | 40 | 40 | 40 | 90 | 60 |
| | (degrees) | vs. Stainless steel | 40 | 40 | 40 | 40 | 40 | 90 | 80 |

| | | | | | | | | |
|---|---|---|---|---|---|---|---|---|
| Evaluation | Droplet fixation to object | good | good | good | good | good | poor | poor |
| as spray | Dye stability | good | good | good | good | good | poor | poor |
| agent, etc. | | | | | | | | |

| | | | Compar- | | | |
|---|---|---|---|---|---|---|
| | | | ative | Exam- | Exam- | Exam- |
| | | | Exam- | ple | ple | ple |
| | | | ple 7-5 | 7-4 | 7-5 | 7-6 |
| Microorganism | Staphylococcus aureus (10⁵ cfu/mL) | | | | | |
| Organic dye | Organic dye | Concentration (μmol/L) in | 20 | 20 | 20 | 20 |
| composition | (A) (Dye of | total organic dye composition | | | | |
| | Formula (4)) | Content (% by mass (of dye solution) | 2 | 2 | 2 | 2 |
| | | (A)/{(A) + (B) + (C)} × 100 | | | | |
| | Alcohol (B): | Content (% by mass) | 40 | 80 | 90 | 98 |
| | Propanediol | (B)/{(A) + (B) + (C)} × 100 | | | | |
| | Water (C) | Content (% by mass) | 58 | 18 | 00 | 0 |
| | | (C)/{(A) + (B) + (C)} × 100 | | | | |
| Fluorescence/ | Fluorescence emission upon light | | fair | fair | fair | fair |
| color change | irradiation (visual visibility) | | | | | |
| upon dye | Color change | | fair | fair | fair | fair |
| hydrolysis | | | | | | |
| Physical | Viscosity (mPa · s) | | 3.6 | 15 | 23 | 33 |
| properties | Contact | vs. Polyethylene | 60 | 50 | 50 | 50 |
| | angle | vs. Polypropylene | 40 | 40 | 40 | 40 |
| | (degrees) | vs. Stainless steel | 60 | 40 | 30 | 30 |

| | | | | | |
|---|---|---|---|---|---|
| Evaluation | Droplet fixation to object | poor | good | good | good |
| as spray | Dye stability | poor | good | good | good |
| agent, etc. | | | | | |

### Test Example 8

### Preparation of Organic Dye Composition

Organic dye compositions of Examples 8-1 to 8-6 and Comparative Examples 8-1 to 8-5 were prepared by using a DMSO solution (excitation wavelength: 490 nm, dye stock solution concentration: 0.1 wt % (1 mg/mL in DMSO)) of an organic dye represented by Formula (2) as the organic dye (A), 1,3-propanediol (boiling point: 213° C.) as the alcohol (B), and pure water obtained by a water production device “milliQ” manufactured by Merck Corporation as the water (C), and blending and stirring the respective components in the amounts (% by mass) shown in Table 8.

Each of the obtained organic dye compositions was used, and the evaluation tests (1) to (5) were performed in the same manner as in Test Example 1 except that Staphylococcus aureus (105 cfu/mL) was used as the bacterial liquid. The results are summarized in Table 8.

TABLE 8

| | | | Compar- | Compar- | | | | Compar- | Compar- |
|---|---|---|---|---|---|---|---|---|---|
| | | | ative | ative | Exam- | Exam- | Exam- | ative | ative |
| | | | Exam- | Exam- | ple | ple | ple | Exam- | Exam- |
| | | | ple 8-1 | ple 8-2 | 8-1 | 8-2 | 8-3 | ple 8-3 | ple 8-4 |
| Microorganism | Staphylococcus aureus (10⁵ cfu/mL) | | | | | | | | |
| Organic dye | Organic dye | Concentration (μmol/L) in | 0.01 | 0.1 | 1 | 10 | 20 | 20 | 20 |
| composition | (A) (Dye of | total organic dye composition | | | | | | | |
| | Formula (2)) | Content (% by mass (of dye solution)) | 2 | 2 | 2 | 2 | 2 | 2 | 2 |
| | | (A)/{(A) + (B) + (C)} × 100 | | | | | | | |
| | Alcohol (B): | Content (% by mass) | 60 | 60 | 60 | 60 | 60 | 0 | 20 |
| | Propanediol | (B)/{(A) + (B) + (C)} × 100 | | | | | | | |
| | Water (C) | Content (% by mass) | 38 | 38 | 38 | 38 | 38 | 98 | 78 |
| | | (C)/{(A) + (B) + (C)} × 100 | | | | | | | |
| Fluorescence/ | Fluorescence emission upon light | | poor | poor | fair | fair | fair | fair | fair |
| color change | irradiation (visual visibility) | | | | | | | | |
| upon dye | Color change | | poor | poor | fair | fair | fair | fair | fair |
| hydrolysis | | | | | | | | | |
| Physical | Viscosity (mPa · s) | | 8 | 8 | 8 | 8 | 8 | 1 | 2 |
| properties | Contact | vs. Polyethylene | 50 | 50 | 50 | 50 | 50 | 90 | 60 |
| | angle | vs. Polypropylene | 40 | 40 | 40 | 40 | 40 | 90 | 60 |
| | (degrees) | vs. Stainless steel | 40 | 40 | 40 | 40 | 40 | 90 | 80 |

| | | | | | | | | |
|---|---|---|---|---|---|---|---|---|
| Evaluation | Droplet fixation to object | good | good | good | good | good | poor | poor |
| as spray | Dye stability | good | good | good | good | good | poor | poor |
| agent, etc. | | | | | | | | |

| | | | Compar- | | | | |
|---|---|---|---|---|---|---|---|
| | | | ative | Exam- | Exam- | Exam- | Exam- |
| | | | Exam- | ple | ple | ple | ple |
| | | | ple 8-5 | 8-3 | 8-4 | 8-5 | 8-6 |
| Microorganism | Staphylococcus aureus (10⁵ cfu/mL) | | | | | | |
| Organic dye | Organic dye | Concentration (μmol/L) in | 20 | 20 | 20 | 20 | 20 |
| composition | (A) (Dye of | total organic dye composition | | | | | |
| | Formula (2)) | Content (% by mass (of dye solution)) | 2 | 2 | 2 | 2 | 2 |
| | | (A)/{(A) + (B) + (C)} × 100 | | | | | |
| | Alcohol (B): | Content (% by mass) | 40 | 60 | 80 | 90 | 98 |
| | Propanediol | (B)/{(A) + (B) + (C)} × 100 | | | | | |
| | Water (C) | Content (% by mass) | 58 | 38 | 18 | 8 | 0 |
| | | (C)/{(A) + (B) + (C)} × 100 | | | | | |
| Fluorescence/ | Fluorescence emission upon light | | fair | fair | fair | fair | fair |
| color change | irradiation (visual visibility) | | | | | | |
| upon dye | Color change | | fair | fair | fair | fair | fair |
| hydrolysis | | | | | | | |
| Physical | Viscosity (mPa · s) | | 3.6 | 8 | 15 | 23 | 33 |
| properties | Contact | vs. Polyethylene | 60 | 50 | 50 | 50 | 50 |
| | angle | vs. Polypropylene | 40 | 40 | 40 | 40 | 40 |
| | (degrees) | vs. Stainless steel | 60 | 40 | 40 | 30 | 30 |

| | | | | | | |
|---|---|---|---|---|---|---|
| Evaluation | Droplet fixation to object | poor | good | good | good | good |
| as spray | Dye stability | poor | good | good | good | good |
| agent, etc. | | | | | | |

### Discussion

As is clear from the results in Tables 1 to 8, it was found that the organic dye compositions of all Examples were effective in the detection of microorganisms because fluorescence emission and color change due to the presence of E. coli and S. aureus could be confirmed. In addition, the droplet was well fixed to an object made of each material of polyethylene, polypropylene, and stainless steel (object to be sprayed), and further, all of the contact angles were 90° or less. Moreover, sufficient storage stability (dye stability) of the organic dye in the organic dye composition could be also confirmed.

As the organic dye (A), the color change upon light irradiation could be more clearly visually recognized by the dye represented by Formula (4) than the dye represented by Formula (2), in the detection of both E. coli and S. aureus. In addition, as the alcohol (B), it was found that the fluorescence emission and color change upon light irradiation can be more clearly confirmed by glycerin than by propanediol.

On the other hand, in the organic dye compositions of Comparative Examples in which the concentration of the organic dye (A) was too low (Comparative Examples 1-1, 1-2, and the like), no microorganism could be detected. In Comparative Examples not containing the alcohol (B) (Comparative Example 1-3 and the like) and Comparative Examples having an excessively low viscosity (Comparative Examples 1-4, 1-5, and the like), the droplet fixation to the object was insufficient, or the dye stability was poor. In addition, the organic dye compositions of Comparative Examples in which the viscosity of the composition was too high (Comparative Example 1-6 and the like) had too high viscosity to be sprayed.

This application is based on Japanese Patent Application No. 2022-121783 filed on Jul. 29, 2022, the contents of which are included in the present application.

In order to express the present invention, the present invention has been described above appropriately and sufficiently through the embodiments with reference to specific examples and the like. However, it should be recognized by those skilled in the art that changes and/or improvements of the above-described embodiments can be readily made. Accordingly, changes or improvements made by those skilled in the art shall be construed as being included in the scope of the claims unless otherwise the changes or improvements are at the level which departs from the scope of the appended claims.

According to the present invention, since the disinfection effect and the presence or absence of microorganisms in a wide range can be easily checked in a short time, the present invention has wide industrial applicability in various technical fields such as an environmental field, a medical field, an inspection field, and a hygiene field.

1. An organic dye composition comprising:
an organic dye (A) that is discolored by hydrolysis and emits fluorescence by excitation light having a wavelength of 400 nm or more and 550 nm or less; an alcohol (B); and water (C),
wherein a content rate of the organic dye (A) is 1 μmol/L or more, and
a viscosity is 5 mPa·s or more and 45 mPa·s or less.

2. The organic dye composition according to claim 1, wherein a contact angle with respect to at least one selected from stainless steel, artificial marble, quartz, resin, and enamel is 90° or less.

3. The organic dye composition according to claim 1, wherein the organic dye (A) is at least one selected from compounds represented by following Formulas (1) to (5):

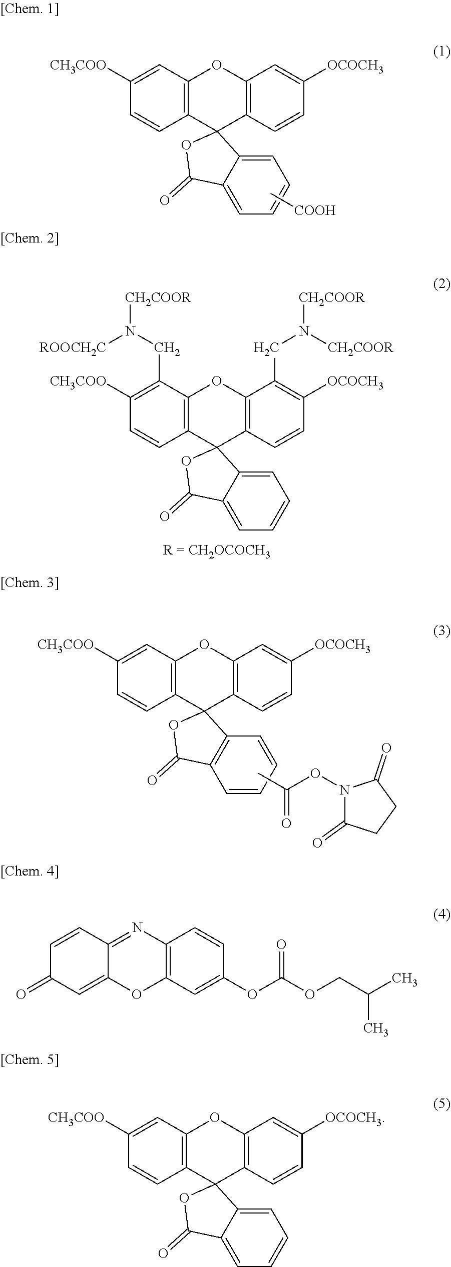

4. The organic dye composition according to claim 1, wherein the alcohol (B) contains an alcohol having a boiling point of 180° C. or higher.

5. The organic dye composition according to claim 4, wherein the alcohol is at least one selected from glycerin and propanediol.

6. The organic dye composition according to claim 4, wherein a content of the alcohol (B) is 50% by mass or more with respect to an entire organic dye composition.

7. A spray agent comprising the organic dye composition according to claim 1.

8. A microorganism visualization method comprising:
attaching, to an object, the organic dye composition according to claim 1; and
confirming presence or absence of discoloration in the object.

9. A microorganism visualization method comprising:
attaching, to an object, the organic dye composition according to claim 1; and
irradiating the object with light having a wavelength of 400 nm or more and 550 nm or less by a light source.

10. The microorganism visualization method according to claim 9, wherein the object is observed through a filter after light irradiation.

11. A microorganism visualization system for attaching, to an object, the organic dye composition according to claim 1, and visualizing microorganisms, the microorganism visualization system comprising:
a light source that irradiates the object with light; an image capturing unit that captures an image of the object to obtain first luminance information; a storage unit that holds second luminance information on the microorganisms; a signal processing unit that compares the first luminance information with the second luminance information and determines the presence or absence of microorganisms; an image generation unit that generates an image from the signal; and a display unit that displays the generated image.
